(19) United States
(12) Patent Application Publication
(10) Pub. No.: US 2026/0123952 A1
(43) Pub. Date: May 7, 2026

(54) METHOD AND APPARATUS FOR TREATMENT OF PULMONARY CONDITIONS

(71) Applicant: AerWave Medical, Inc., Stony Brook, NY (US)

(72) Inventors: Reinhard J. Warnking, Westlake, FL (US); Robert Pacheco, Weehawken, NJ (US); Subash Shrestha, Lake Ronkonkoma, NY (US)

(73) Assignee: AerWave Medical, Inc., Stony Brook, NY (US)

(21) Appl. No.: 19/118,806

(22) PCT Filed: Oct. 6, 2023
(86) PCT No.: PCT/US2023/034606

§ 371 Date: Apr. 7, 2025

Related U.S. Application Data
(60) Provisional application No. 63/416,097, filed on Oct. 14, 2022.

(51) Int. Cl. A61B 17/32; A61B 8/08; A61B 8/12; A61B 17/00; A61B 17/24

(52) U.S. Cl. CPC A61B 17/320068; A61B 8/085; A61B 8/12; A61B 2017/00026; A61B 2017/00557; A61B 2017/242; A61B 2017/320069

(57) ABSTRACT

Apparatus and methods for deactivating bronchial nerves and ablating smooth muscle extending along a bronchial branch of a mammalian subject to treat asthma and related conditions. An ultrasonic transducer (11) is inserted into the bronchus as, for example, by advancing the distal end of a catheter (10) bearing the transducer into the bronchial section to be treated. The ultrasonic transducer emits ultrasound so as to heat tissues throughout a relatively large impact volume (13) to a temperature sufficient to inactivate nerve conduction and ablate smooth muscle but insufficient to cause rapid ablation or necrosis of the surrounding tissues. The treatment can be performed without locating or focusing on individual bronchial nerves or smooth muscle.

### BACKGROUND

This application claims priority to provisional application Ser. No. 63/416,097 filed Oct. 14, 2022, the entire contents of which are incorporated herein by reference.

### FIELD OF THE INVENTION

This invention relates to apparatus and methods for the treatment of pulmonary conditions such as asthma and COPD.

### BACKGROUND OF RELATED ART

Successful treatment of pulmonary diseases such as asthma is important since these diseases represent a significant global health issue with reduced quality of life. While drug therapy (Bronchodilators, Anti Inflammatories and Leukotrines Modifiers) can be used to treat asthma, it is not always successful and very expensive. Asthma is a disorder that is characterized by airway constriction and inflammation resulting in breathing difficulties. Wheezing, shortness of breath and coughing are typical symptoms.

These symptoms are caused by increased mucus production, airway inflammation and smooth muscle contraction resulting in airway obstruction. This obstruction can be treated by injuring and scarring the bronchial walls. This remodeling of the bronchial walls stiffens the bronchia and reduces contractility. Mechanical means and heat application have been proposed as in U.S. Pat. No. 8,267,094 B2. Other approaches focus on destruction of smooth muscle cells surrounding the bronchia as described in U.S. Patent No. 2012/0143099A1 and U.S. Pat. No. 7,906,124B2. Others describe applying RF energy to the bronchial wall and thereby directly widening the bronchia through a process such as disclosed in U.S. Pat. No. 7,740,017B2 and U.S. Pat. No. 8,161,978B2. Whatever the process, the bronchial wall will be damaged, and the procedure therefore has to be staged in order to limit damage and side effects as described in U.S. Pat. No. 7,740,017B2. European Patent No. EP2405841 describes applications of heat shocks through infused agents. Inactivating conduction of the nerves surrounding the bronchia has been proposed in U.S. Patent Publication No. 2012/0203216A1 through mechanical action i.e., puncturing, tearing, cutting nerve tissue.

In other proposed or extant procedures, nerve tissue ablation is implemented by applying energy (RF, HIFU, Microwave, Radiation and Thermal Energy) directly to the nerves percutaneously. However, it is not taught how to identify the nerve location in order to align the energy focal zone (i.e., HIFU) with the nerve location. This is an issue since nerves are too small to be visualized with standard ultrasound, CT or MRI imaging methods. Therefore, the focal zone of the energy field cannot be predictably aligned with the target or nerve location. U.S. Pat. No. 8,088,127B2 teaches to denervate by applying RF energy to the bronchial wall with the catheter positioned inside the bronchial lumen. It is proposed to protect the bronchial wall through simultaneous cooling of the wall. This of course makes the device structure complicated and bulky and therefore difficult to deliver through a bronchoscope working channel. Also, the RF ablation is limited to the electrode contact area which requires adding together several energy applications to create a circumferential treatment volume. Numerous ablation sectors need to be pieced together to obtain a circumferential ablation zone with increased probability of affecting nerves. Due to catheter size and the need for multiple energy applications per bronchus, denervation with a cooled RF ablation device is practically limited to denervation in the left and right main bronchi in order to keep the overall number of energy applications low (at least 4 per bronchus) and therewith the procedure time acceptable. However, this main bronchial location carries the risk of esophageal and peri esophageal nerve damage which complicates the procedure further, requiring fluoroscopic monitoring of the distance between ablation and an esophageal marker-balloon. How to safely simplify lung denervation procedures by employing circumferential ultrasound in secondary bronchi is described in U.S. patent application Ser. No. 17/350,848, U.S. Patent Publication No. 2021/0316161.

The sectorial RF ablation in main bronchi is not only complicated and time consuming but often also limited as far as efficacy is concerned because often RF energy delivery needs to be limited by reducing RF power or lesion geometry, i.e., foregoing posterior ablation segments to avoid damaging peri esophageal nerves or the esophagus located in the vicinity of the posterior section off the main bronchi. As described in U.S. Patent Application Publication No. 2021/0316161, efficacy of ultrasound denervation in secondary bronchi will not be negatively affected by these safety measures. However, there is a need for a device and method to increase efficacy further.

In order to explain the difficulties associated with accomplishing this task without causing other damage, the anatomy of the bronchial system and nerves will now be described. Shown in FIG. 6 is an illustration of the bronchial tree (1). FIG. 3 shows a cross section of a bronchial tube surrounded with smooth muscle (7) and nerves (6). In addition, FIG. 5 shows a longitudinal section of a bronchus (1) and the adjacent nerves (6). As can be seen from these two FIGS. (3 and 5), the bronchial nerves (6) surround the bronchial tubes. Different individuals have the nerves (6) in different locations around the bronchial tubes. Thus, the nerves may be at different radial distances from the central axis where the energy emitter (11) is placed (FIG. 3). The nerves (6) also may be at different locations around the circumference of the bronchial tubes as shown in FIG. 3. It is not practical to locate the bronchial nerves by referring to anatomical landmarks. Moreover, it is difficult or impossible to locate individual bronchial nerves using common in vivo imaging technology.

The inability to locate and target the bronchial nerves (6) makes it difficult to disconnect the bronchial nerve activity using non-surgical techniques without causing damage to the bronchial walls or causing other side effects. For example, attempts to apply energy to the bronchial nerves can cause effects such as stenosis and necrosis of adjacent tissues. In addition, the inability to target and locate the bronchial nerves (6) makes it difficult to ensure that bronchial nerve activity has been discontinued enough to achieve an acceptable therapeutic treatment.

As noted above, U.S. Pat. No. 8,088,127B2 suggests the use of a radio frequency (“RF”) emitter connected to a catheter, which is inserted in the bronchial tree. The RF emitter is placed against the bronchial wall and the RF energy is emitted to heat the nerves to a temperature that reduces the activity of bronchial nerves which happen to lie in the immediate vicinity of the emitter. In order to treat all the nerves surrounding the bronchial tubes, the RF emitter source must be repositioned around the inside of each bronchial tube section multiple times. In order to protect the bronchial wall, this RF heat application is combined with a cooling application which makes the procedure more complicated. The emitter may miss some bronchial nerves, leading to an incomplete treatment. Moreover, the RF energy source (electrode) must contact the bronchial wall to be able to heat the surrounding tissue and nerves, which may cause damage or necrosis to the inner lining of the bronchi. Since each denervation consists of several segmental RF applications and the cooled RF treatment catheter is rather bulky, RF denervation is practically limited to main bronchial locations. This in turn, due to the vicinity of the esophagus, requires safety measures like placement of an esophageal marker balloon and fluoroscopic imaging to monitor the distance between marker and treatment balloons. If a safe distance between marker and treatment balloons cannot be achieved, ablation energy needs to be limited or posterior ablation segments need to remain untreated in order to protect the esophagus and peri esophageal nerves from damage.

WO 2007/009118 also suggests the use of high intensity focused ultrasound to deactivate the bronchial nerves. It is not clear how a High Intensity Focused Ultrasound zone can be aligned with the targeted bronchial nerves. It is difficult or impossible to align this highly focused zone with the bronchial nerves because it is difficult or impossible to visualize and target the bronchial nerves with current technology, and because the bronchial nerves may lie at different radial distances and circumferential locations from the central axis of bronchi. The latter problem is aggravated in patients who have bronchi with large variations in shape or thickness. Moreover, the small focal zone can encompass only a small segment of each bronchial nerve along the lengthwise direction of the bronchi. Since nerves tend to re grow, a small treatment zone allows the nerves to reconnect in a shorter period of time.

The need exists for an efficient and effective device and method to deactivate the bronchial nerves without causing damage or unwanted effects on other tissue.

### BRIEF SUMMARY OF THE INVENTION

The present invention overcomes the problems and deficiencies of the prior art. Various devices and methods of the present invention are disclosed herein which provide an efficient and effective device and method to deactivate the bronchial nerves without causing damage or unwanted effects on other tissue. Furthermore, several of the features of the devices and methods disclosed herein can have application for other treatments.

Features of the system, device and methods of the present invention for use in pulmonary treatment include generally one or more of the following: 1) ultrasonic energy emitted at a therapeutic level to provide a therapeutic mode to inactivate conduction of bronchial nerves; 2) ultrasonic energy emitted in a sub-therapeutic level to provide a diagnostic mode for a) dosing optimization; b) positioning; c) coupling and/or bronchial diameter determination; and/or d) impedance measurements for cartilage detection for device positions. Each of these are discussed in more detail below.

Additionally, further features of the system, device and method can include one or more of the following i) pulsating cooling fluid within the balloon for diameter measurements; ii) pulsating cooling fluid within the balloon to measure bronchial compliance and ablation; iii) utilization of ultrasound energy at a sub-therapeutic level to determine tissue type or state; and/or iv) utilization of ultrasound energy at a sub-therapeutic level to assess treatment. Each of these are discussed in more detail below.

It should be appreciated that the devices and methods of the present invention need not incorporate all of the above features 1-2(a-d) or all of the above features i-iv to provide clinical advantages over the prior art. Therefore, the devices and methods in various embodiments of the present invention can have one of the features 1-2 (a-d) and/or i-iv, two of the features 1-2(a-d) and/or i-iv, etc.

In accordance with one aspect of the present invention, a system including an apparatus is provided for inactivating bronchial nerve conduction combined with the ablation of smooth muscle in a human or non-human mammalian subject. The present invention contemplates a simpler and safer solution to treating adverse pulmonary conditions, as compared to performing denervation by sectional RF ablations, and such treatment causes little or no damage to bronchial walls and surrounding tissues. Treatments pursuant to the present invention are much easier and faster to perform than RF techniques. Conventional multiple airway smooth muscle (ASM) ablations (see U.S. Pat. No. 7,740,017B2 and Alair System description, BSX) can be reduced to a single treatment much better tolerated by the patient. Instead of denervation in main bronchi requiring safety measures like esophageal marking under fluoroscopy and electrical and geometrical lesion reduction, the present invention in this aspect combines denervation and airway smooth muscle (ASM) ablation. In preferred embodiments, the invention enables treatment in distal central airways of the third generation (tertiary bronchi) and the fourth generation, to perform ASM ablation in these strategically important central airways combined with denervation to disable ASM contraction in the more distal airways. The major source of airflow resistance is in 3rd and 4th generation bronchi. This will allow one to reduce a 3-session smooth muscle ablation procedure to be performed in one treatment session. This is not contemplated by the prior art, nor achievable, in part because such prior art techniques as RF denervation require devices that are too bulky for disposition in these smaller air passageways while prior art RF SMA ablation devices do not allow to perform denervation.

With a circumferential ultrasound catheter of the present invention, a one-shot 360 degree ablation zone is accomplished so that more distal treatment sites in tertiary and fourth-generation bronchi can be selected without increasing the number of energy applications to impractical levels, as described in U.S. Patent Publication No. 2021/0316161 for denervation in secondary bronchi. In accordance with the present invention, denervation in the lobar central airways is realistic and practical since only one energy application per bronchus is required compared to at least 4 with RF segmental ablation techniques.

Furthermore, the physical properties of ultrasound heating allow a miniaturized structure of the ultrasound catheter with the cylindrically radiating energy source located at its center. This allows for the ultrasound catheter of the present invention to perform smooth muscle ablation combined with denervation in 3rd and 4th generation bronchi which will provide for a more effective treatment than smooth muscle ablation (SMA) or lung denervation alone.

The apparatus according to the present invention preferably includes an ultrasound transducer adapted for insertion into the bronchial system of the mammalian subject. The ultrasound transducer preferably is arranged to transmit circumferential ultrasound energy. The apparatus preferably also includes an actuator or control unit (e.g., programmed microprocessor or hard-wired logic circuit) electrically connected to the transducer. The actuator preferably is configured/adapted to control through time and power variations the ultrasound transducer to transmit ultrasound energy into a circumferential impact volume encompassing a target bronchial branch or tube so that the ultrasound energy is applied at a therapeutic level sufficient to inactivate conduction of bronchial nerves and ablate smooth muscle throughout the impact volume. Given the wide variation of bronchial diameters, this procedure is realistic by combining the therapeutic operation with diagnostic measurements, e.g., volumetric A mode measurements to determine the diameter and adjust the therapeutic ultrasound energy accordingly, as described in U.S. Patent Publication 2021/0036161.

In the present invention, the ultrasound transducer is preferably contained in a compliant or expandable balloon filled with a coupling fluid. The actuator or control unit is configured to control the temperature of the fluid, for instance, via a heating element and/or a cooling coil, so that the temperature induced in the smooth muscle and nerves by the application of ultrasound energy in the impact volume may be maximized, without unduly damaging the mucosa and wall of the bronchial tube at the locus of the treatment. The apparatus may include a temperature sensor, for instance in the balloon, to ensure that the coupling fluid maintains a level of thermal energy that is safe for the bronchial tube tissue and mucosa.

The apparatus may further include a catheter with a distal end and a proximal end, the catheter being provided at the distal end with the compliant balloon and the transducer being mounted to the catheter adjacent the distal end. The compliant balloon can be expandable by pressurizing the coupling fluid and is configured to make contact with the bronchial wall. The coupling fluid is preferably a circulating cooling fluid that serves in part to conduct ultrasound energy from the transducer to the bronchial walls and surrounding tissue and nerves. Pursuant to its cooling function, the coupling fluid transports excessive heat away from the transducer and protects the bronchial lining from injury. (About half of the electrical energy supplied to the transducer is converted into heat while the other half is converted to ultrasonic energy). Another function of the pressurized coupling fluid can be to monitor bronchial compliance and therewith SMA ablation. By monitoring the cooling fluid pressure and in case of a peristaltic pump the pressure and balloon pulsation, the compliance and therewith the degree of smooth muscle ablation of the surrounding bronchus can be characterized. If smooth muscle is ablated, the vessel wall tends to be very compliant and follows the pulsation. With intact smooth muscle, the pulsation is dampened. The transducer is adapted to transmit the ultrasound energy in a 360° cylindrical pattern surrounding a transducer axis, and the catheter may be constructed and arranged to hold the axis of the transducer generally parallel to the axis of the bronchial tube. This can be achieved by the symmetric expandable balloon which provides a 360° contact and thus spacing from the bronchial wall and the use of ultrasound signal echoes to ensure continuous wall engagement.

The actuator or control unit of the present invention in some embodiments is configured not only to activate the transducer to emit therapeutic ultrasound but also to operate the transducer in a diagnostic mode to generate a volumetric A mode signal, integrating ultrasound echoes throughout the treatment volume, which is analyzed to (i) ensure circumferential coupling; (ii) measure the mean bronchial diameter to optimize dosing (power, time) for the relatively deep denervation and very shallow SMA ablation and in the case of denervation in secondary bronchi; (iii) determine tissue type; (iv) assess treatment and/or (v) ensure inter cartilage positioning as described in U.S. Patent Publication No. 2021/0316161.

A method according to one aspect of the present invention includes the steps of inserting an ultrasound transducer into a central airway of a 3rd and/or 4th generation bronchus of the subject and operating the actuator or control unit to activate the transducer to transmit therapeutically effective ultrasound energy into a circumferential impact volume encompassing the 3rd and/or 4th generation bronchus. The actuator or control unit controls the applied ultrasound energy to inactivate conduction of all the nerves in the impact volume and simultaneously ablate smooth muscle within the treatment volume. Typical bronchial 3rd and 4th generation diameters range around 5 mm. Alternatively, in larger bronchi, nerves and smooth muscle can be targeted separately by optimizing the dosing parameters between shallow smooth muscle volume and relatively deep nerve volume. For example, the actuating of the transducer may be implemented so as to maintain the temperature of the bronchial wall below about 45° C. while heating the solid tissues in particular airway smooth muscle (ASM) within the impact volume, and/or the bronchial nerves in the impact volume, to at least about 60° C. As discussed above, the actuator or control unit is preferably configured to maintain the temperature of the bronchial wall at a temperature below 45° C. in part by ensuring a sufficiently low temperature of the coupling fluid in the balloon.

Because the impact volume is relatively large, and because the tissues throughout the impact volume preferably reach temperatures sufficient to inactivate nerve conduction and cause smooth muscle ablation, the preferred methods according to this aspect of the invention can be performed successfully without precisely determining the actual locations of the bronchial nerves or smooth muscle fibers. The treatment can be performed without measuring the temperature of electrodes and adjacent tissues as described in the literature and patents to Athmatx. Based on the volumetric A mode bronchial diameter measurement, the therapeutic dose (power, time) will be optimized for each anatomical situation. Moreover, the treatment preferably is performed without causing injury to the mucosa. The system/apparatus of the present invention can be used to inactivate relatively long segments of smooth muscle and bronchial nerves, so as to reduce the possibility of nerve or smooth muscle recovery which would re-establish contraction along the inactivated smooth muscle segments and distal of the ablated nerve segments.

The present invention also contemplates combined smooth muscle ablation and denervation in 3rd and 4th generation bronchial segments. The invention provides probes which can be used in the method and apparatus discussed above, and apparatus incorporating structure/features for performing the steps of the methods discussed above.

### BRIEF DESCRIPTION OF THE DRAWINGS

So that those having ordinary skill in the art to which the subject invention appertains will more readily understand how to make and use the apparatus (device) disclosed herein, preferred embodiments thereof will be described in detail hereinbelow with reference to the drawings, wherein:

FIG. 1 is an anatomical view of typical main bronchi BR and BL and associated structures and the apparatus and system of the present invention controlling the ablation.

FIG. 2 shows a treatment catheter of the present invention advanced through a bronchoscope into the right bronchial branch and a diagrammatic sectional view depicting the circumferential ultrasound treatment volume or impact zone (in phantom).

FIG. 3 shows a cross section through a bronchial tube with smooth muscle and nerves with an ultrasound transducer of the catheter of FIG. 2 in the center surrounded by the cooling fluid in the compliant balloon.

FIG. 4 is partially a graph showing a volumetric A mode signal for diameter measurement and inter-cartilage transducer placement and partially a bronchial tube cross-sectional view, with arrows indicating cartilage structures giving rise to respective artifacts of the volume integrated A mode signal.

FIG. 5 shows a right bronchial branch with adjacent nerves running alongside the bronchial tube.

FIG. 6 shows a bronchial tree in its entirety.

FIG. 7 is a graph of tissue temperature as a function of distance from an ultrasound transducer and time of transducer activation, providing an example for temperature profiles and resulting treatment depths for denervation determined by acoustic power and time optimized for a bronchial diameter of 10 mm and a cooling fluid temperature of 20 deg C.

FIGS. 8A and 8B are side elevational views of two devices having respective electrode configurations for cartilage detection through electrical impedance measurements, showing disposition of the devices inside a bronchus with cartilage rings.

### DETAILED DESCRIPTION

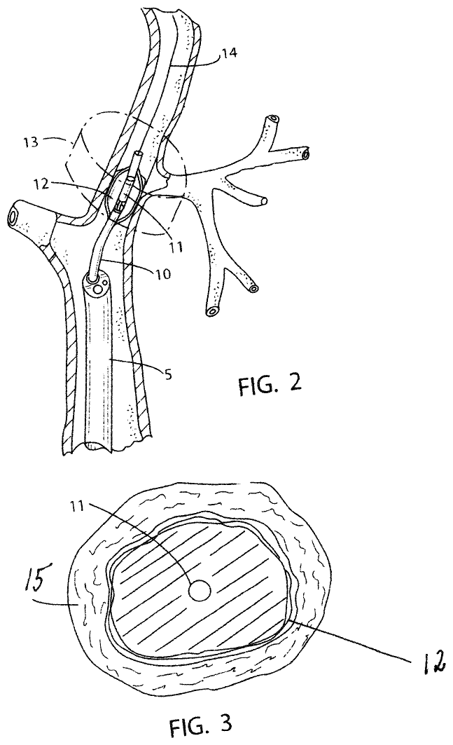

Referring now to the drawings and particular embodiments of the present disclosure, wherein like reference numerals identify similar structural features of the apparatus throughout the several views, an apparatus according to one embodiment of the invention is illustrated in FIG. 2 and includes an elongated tubular member in the form of a catheter 10, advanced through the working channel of a bronchoscope 5. Alternatively, the catheter 10 can be advanced through a sheath or directly without any delivery instrument over a guide wire 14 which has been placed by electromagnetic navigation bronchoscopy (ENB). The sheath, generally, may be in the form of an elongated tube having a proximal end, a distal end and a proximal-to-distal axis. As used in this disclosure with reference to elongated elements for insertion into the body, the term “distal” refers to the end which is inserted into the body first, i.e., the leading end during advancement of the element into the body, whereas the term “proximal” refers to the opposite end which is closer to the clinician. The sheath may be a steerable sheath. Thus, the sheath may include known elements such as one or more pull wires (not shown) extending between the proximal and distal ends of the sheath and connected to a steering control arranged so that actuation of the steering control by the operator flexes the distal end of the sheath in a direction transverse to the axis. The scope can also be steerable.

Catheter 10 has a proximal end, a distal end and a proximal-to-distal axis which is located preferably coincident with the bronchial axis during a treatment procedure.

Catheter 10 has a compliant or alternately collapsible and expandable balloon 12 mounted at the distal end. In an inflated condition (FIGS. 2 and 3), balloon 12 engages the bronchial wall and therewith allows for ultrasound to be conducted into the bronchial wall and surrounding tissues from a transducer 11 located inside the balloon 12.

Ultrasound transducer 11 (FIG. 3) is mounted adjacent the distal end of catheter 10 within balloon 12. Transducer 11, which is preferably formed from a ceramic piezoelectric material, is of a tubular shape and has an exterior emitting surface in the form of a cylindrical surface of revolution about the proximal-to-distal axis of the transducer 11. The transducer 11 typically has an axial length of approximately 2-10 mm, and preferably about 6 mm, although other dimensions and multiplanar configurations are also contemplated. The outer diameter of the transducer 30 is approximately 1.5-3 mm in diameter, and preferably about 2 mm, although other dimensions are also contemplated. The transducer 11 also has conductive coatings (not shown) on its interior and exterior surfaces. Thus, the transducer may be physically mounted on a metallic support tube (not shown) which in turn is mounted to the catheter 10. The coatings are electrically connected to ground and signal wires. Wires (not illustrated) extend from the transducer 11 through a lumen in the catheter shaft to a connector electrically coupled with the ultrasound system. The lumen extends between the proximal end and the distal end of a catheter 10, while the wires extend from the transducer 11, through the lumen, to the proximal end of the catheter 10.

Transducer 11 is arranged so that ultrasonic energy generated in the transducer is emitted principally from the exterior emitting surface. Thus, the transducer may include features designed to reflect ultrasonic energy directed toward the interior of the transducer so that the reflected energy reinforces the ultrasonic vibrations at the exterior surface. For example, support tube and transducer 11 may be configured so that the energy emitted from the interior surface of the transducer 11 is reflected back to enhance the overall efficiency of the transducer. In this embodiment, the ultrasound energy generated by the transducer 11 is reflected at the interior mounting to reinforce ultrasound energy propagating outwardly from the transducer 11, thereby ensuring the ultrasound energy is directed towards target tissues from an external surface of the transducer 11.

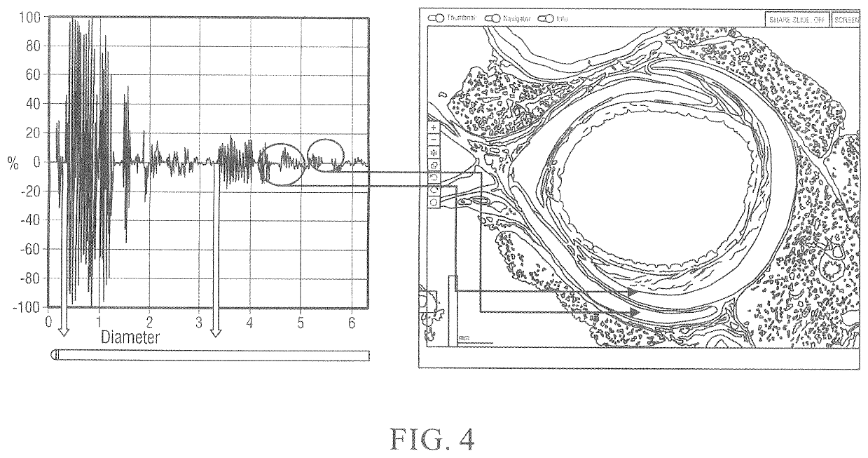

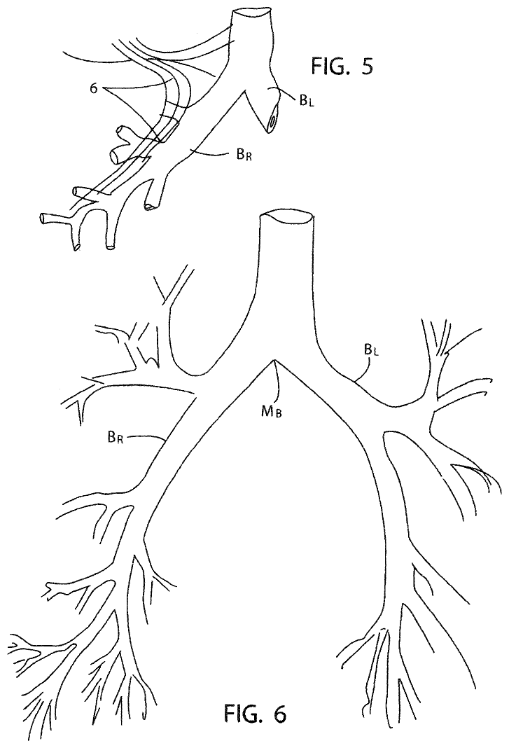

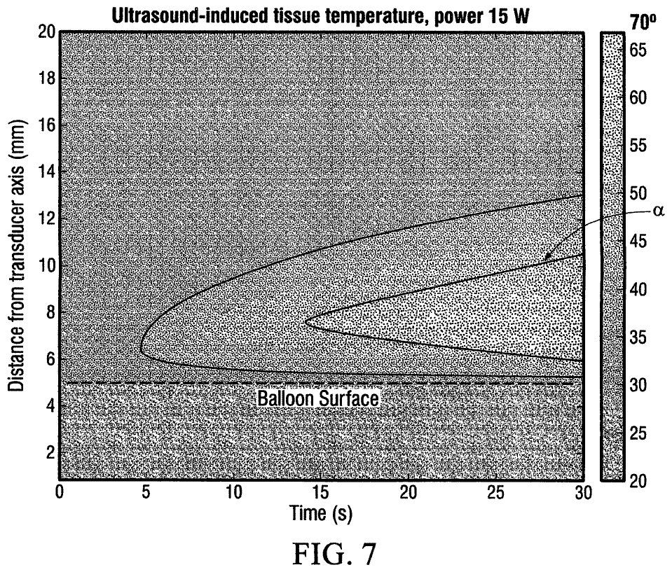

Transducer 11 is also arranged to convert ultrasonic echoes or waves reflected from organic structures and impinging on the exterior surface into electrical signals on wires as shown in FIG. 4. If a reflecting structure such as a bronchial wall is not perfectly circular, the widths of the reflected signal will represent the difference between a maximum diameter of the reflecting structure, d max, and a minimum diameter d min. Stated another way, transducer 11 can act either as an ultrasonic emitter or an ultrasonic receiver. The receiving mode is of particular importance for optimizing the therapeutic impact volume through power and time adjustments based on the bronchial diameter measurement as indicated in FIG. 7. FIG. 4 shows an example for a volume integrated A mode signal and the diameter determination based on the echo analysis which is used to ensure effective denervation and smooth muscle ablation (SMA) while minimizing collateral damage. Also shown are typical volumetric A mode cartilage echo signatures.

It is to be noted that transducer 11 is operatively connected to an actuator or control unit that provides both low-power diagnostic (diagnostic mode) and high-power therapeutic electrical activation signals (therapeutic mode) to the transducer, at different times. The actuator or control unit may be programmed or hard-wired in this diagnostic mode to calculate and select intensity and duration of outgoing therapeutic ultrasonic waveforms (as well as control of coupling fluid temperature). The actuator or control unit may be additionally programmed or hard-wired to interpret volumetric A mode diagnostic echoes, for instance, to determine adequate balloon-bronchus contact and longitudinal cartilage tissue locations, as well as to ascertain radial diameters of different tissues, especially the bronchial wall for dosimetry purposes, that is, to inform the selection of therapeutic activation power level (intensity) and duration.

The transducer 11 is designed to operate, for example, at a frequency of approximately 1 MHz to approximately a few tens of MHz, and typically at approximately 10 MHz. The actual frequency of the transducer 11 typically varies somewhat depending on manufacturing tolerances. The optimum actuation frequency of the transducer may be encoded in a machine-readable or human-readable element (not shown) such as a digital memory, bar code or the like affixed to the catheter. Alternatively, the readable element may encode a serial number or other information identifying the individual catheter, so that the optimum actuation frequency may be retrieved from a central database accessible through a communication link such as the internet.

In some embodiments, AI can be used to help improve the accuracy of interpretation of the A Mode signals and/or for therapeutic mode interpretation and analysis for ultrasound parameters.

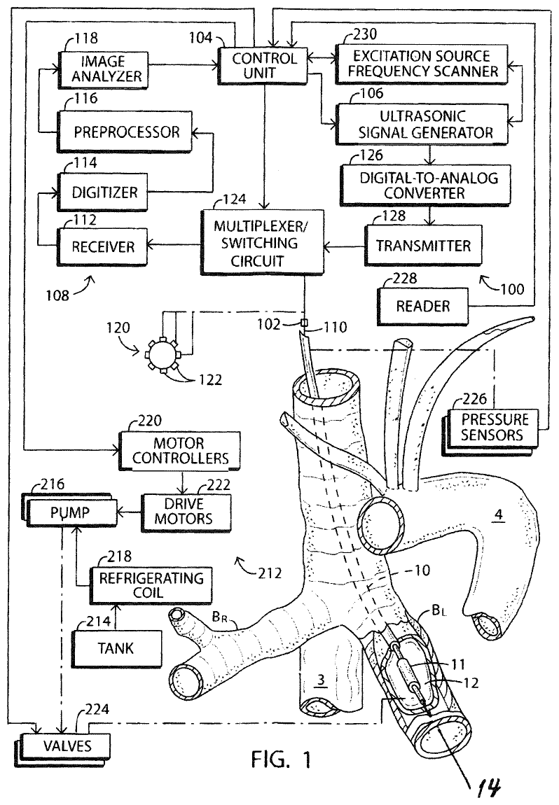

An ultrasound system including an actuator or control unit 104 is releasably connected to catheter 10 and transducer 11 through a plug connector 102 (FIG. 1). The actuator or control unit 104 is configured to effect the energy application and functions described herein.

As discussed above, the ultrasound system includes an ultrasound excitation source or ultrasonic signal or waveform generator 106 configured to control the amplitude and timing of outgoing electrical signals so as to control the power level and duration of the ultrasound signals emitted by transducer 11 to optimize the therapeutic window as indicated in FIG. 7. The excitation source is also arranged to detect electrical volumetric A mode signals as shown in FIG. 4 generated by transducer 11 and appearing on wires and communicate such signals to the control unit.

An energization circuit 100 including control unit 104 and ultrasonic signal generator 106 also includes a detection subcircuit 108 arranged to detect electrical signals generated by transducer 11 and transmitted via wires 110 and communicate such signals to the control unit 104. More particularly, detection subcircuit 108 includes a receiver or echo signal extractor 112, a digitizer 114, an ultrasonic echo signal preprocessor 116, and an image analyzer 118 connected in series to one another. Ultrasonic signal generator 106 produces both therapeutic denervation or ablation signals, e.g., tumor ablation signals, and outgoing diagnostic A mode signals. As discussed hereinafter, the outgoing diagnostic signals and the returning echo signals may be transmitted and picked up by transducer 11. A multiplexer or switching circuit 124 is operated by control unit 104 to switch to a receiving mode after diagnostic signals are emitted during a transmitting mode via a digital-to-analog converter 126 and a transmitter module 128.

In the foregoing embodiments, the diagnostic mode is performed first, following by application of the therapeutic mode. In alternate embodiments, the therapeutic mode can be interleaved with the diagnostic mode and provided in a form of alternate patterns. For example, a therapeutic mode with pulsed signals can be applied intermittently with the diagnostic mode, at equal or unequal intervals, as desired, in a quasi-simultaneous method.

In some embodiments, the diagnostic mode can further be used to determine a tissue type, e.g., ablated/non-ablated or tissue state. That is, ultrasonic energy can be emitted at a sub-therapeutic level as described herein for assessment. This can be achieved since tissue state varies echo amplitudes and frequencies. Ablated tissue typically is more reflective resulting in larger echo amplitudes of shorter duration vs. non ablated soft tissue with smaller echo amplitudes of lower frequency content. Anatomical structures like cartilage and vessels generate typical echo patterns characterized by a leading-edge echo followed by an echo free zone due to the high ultrasound absorption of cartilage. Diameter measurements can be enhanced by amplitude modulation through the balloon pulsation caused by a pulsating pump.

As depicted in FIG. 1, a circulation device 212 is connected to lumens (not shown) within catheter 10 which in turn communicate with balloon 12. The circulation device 212 is arranged to circulate a liquid, preferably an aqueous liquid, through the catheter 10 to the transducer 11 in the balloon 12. The circulation device 212 may include elements such as a tank 214 for holding the circulating coolant, pump(s) 216, a refrigerating coil 218, or the like for providing a supply of liquid to the interior space of the balloon 12 at a controlled temperature, preferably at or below body temperature. The control unit 104 interfaces with the circulation device 212 to control the flow of fluid into and out of the balloon 12, thereby effectuating balloon expansion and contraction. By lowering the coolant temperature, the inner radius of the circumferential treatment volume can be increased in order to protect certain structures like the inner bronchial lining from harmful temperatures. The control unit 104 interfaces with the circulation device 212 to control the flow of fluid into and out of the balloon 12. For example, the control unit 104 may include motor control devices 220 linked to drive motors 222 associated with pumps 216 for controlling the speed of operation of the pumps. Such motor control devices 220 can be used, for example, where the pumps 216 are positive displacement pumps, such as peristaltic pumps. Alternatively, or additionally, the control unit 104 may include structures such as controllable valves 114 connected in the fluid circuit for varying resistance of the circuit to fluid flow (not shown). Also contemplated is using two pumps, one in and one out, to maintain higher flow rates for higher cooling while maintaining reduced balloon pressure.

The ultrasound system may further include one or more pressure and/or flow sensors 226 (FIG. 1) to monitor the liquid pressure and/or flow through the catheter 10 and in another application determine bronchial compliance or flexibility and therewith ASM ablation. At least one pressure sensor or flow sensor 226 monitors the respective pressure or flow of the liquid to the distal end of catheter 10 to determine if there is a blockage, while another sensor 226 monitors leaks in the catheter 10. (Note that in some instances pressure can be controlled by flow). While the balloon 12 is in an inflated state, the pressure sensors 226 maintain a desired pressure in the balloon preferably so that the compliant balloon occludes the bronchus which is controlled through analysis of the volumetric A-mode signal shown in FIG. 4. The control unit 104 is operatively connected to the refrigerating coil 218 (and optionally a heating coil, not separately shown) of the coupling-fluid circulation device 212 for fine tuning the temperature of the liquid in the balloon 12.

The ultrasound system incorporates a reader 228 for reading a machine-readable element on catheter 10 and conveying the information from such element to the control unit or board 104. As discussed above, the machine-readable element on the catheter may include information such as the operating frequency and efficiency of the transducer 11 in a particular catheter 10, and the control unit 104 may use this information to set the appropriate frequency and a power range for exciting the transducer. Alternatively, the control unit 104 may be arranged to actuate an excitation source or frequency scanner 230 to measure the transducer operating frequency by energizing the transducer at a low power level while scanning the excitation frequency over a pre-determined range of frequencies for example 8.5 Mhz 10.5 Mhz, and monitoring the response of the transducer 11 to such excitation and to select the optimal operating frequency. Also, as discussed herein the control unit/system can also monitor the A mode response across frequencies when coupled to see absorption/reflection affects of tissue across frequencies to help determine tissue types and states.

The ultrasonic system may be similar to that disclosed in U.S. Patent Publication No. 2016/0008636, entitled “Ultrasound Imaging Sheath and Associated Method for Guided Percutaneous Trans-Catheter Therapy” the disclosure of which is incorporated by reference herein. Other ultrasonic systems can also be utilized.

After preparing a human or non-human mammalian subject (such as to create a tracheal access site) and connecting the catheter 10 to the ultrasound system and the transducer 11 to the control unit 104, the ultrasound catheter 10 is inserted into the working channel of a bronchoscope after the bronchoscope has been advanced to the desired treatment site under visual guidance via a bronchoscope camera or optical fiber. Alternatively, a steerable sheath, preferably with ultrasound imaging capability as described in U.S. Patent Publication No. 2016/0008636, can be used as a delivery channel for the treatment catheter. In another embodiment the treatment catheter 10 is equipped with a steering or deflection mechanism and can be advanced directly to the treatment site as shown in FIG. 1. If the catheter 10 combines imaging and therapeutic capabilities as described in the '636 patent publication, this delivery method enables the fastest procedure time and is easily tolerated by the patient. Yet another embodiment provides for a guide wire 14 (FIGS. 1 and 2) to be delivered through the working channel of the bronchoscope to the treatment site and the ultrasound treatment catheter 10 to be advanced over the wire after the bronchoscope has been withdrawn. This technique will allow for very small, flexible bronchoscopes to be utilized.

Once the distal end of the catheter 10 is in position within a bronchial branch, pump 216 brings balloon 12 to an inflated condition as depicted in FIGS. 2 and 3. Circumferential contact by the balloon will be ensured through analysis of the volumetric A mode signal. In case of a peristaltic pump, amplitude and signal width fluctuations will allow to identify the balloon/wall echo within the multitude of volume integrated A mode signals. The pulsating waterflow will modulate balloon/tissue coupling and therewith the amplitude/time/phase of the volume integrated A mode signal caused by the circumferentially integrated balloon/tissue reflection. (The sample rate of the signal may also influence the perceived amplitude fluctuations). A controlled fluctuating pulsed flow can be used, such as provided naturally by a peristaltic style pump, and can provide one or more of the following benefits 1) highlighting the balloon wall echo to help determine diameter; 2) helping determine absolute wall compliance, indicating the state of tissue (thickness, etc.); 3) showing relative compliance, such as before and after ablation; and/or 4) indicating relative amount of flow (by showing relative speed of pulsations).

The balloon/tissue echo will change amplitude as well as width synchronous with the waterflow/balloon pulsation. In this condition, the compliant balloon 12 engages the bronchial wall, and thus centers transducer 11 within the bronchial branch, with the axis of the transducer 11 approximately coaxial with the axis of the bronchial branch. This not only provides for a relatively homogeneous energy distribution circumferentially, but also keeps the very high energy levels close to the transducer located inside the cooling fluid where they are harmless, since ultrasound does not interact with fluid. If these peak energy levels were allowed near the bronchial wall (1), injury would result. Another advantage of proper centering is that the treatment volume coincides with the relatively flat portion of the 1/r curve, providing an almost constant power level throughout the treatment volume.

During treatment, the circulation device 212, including pump 216, coils 218, and valves 224 (FIG. 1), maintains a flow of cooled aqueous liquid into and out of balloon 12, so as to cool the transducer 11. The cooled balloon also tends to cool the interior surface of the bronchus. The combination of refrigeration coil 218 and heating coil (not shown) in the circulation device 212 facilitates a fine tuning of the temperature at the balloon-bronchus interface and concomitantly a maximizing of ultrasound-induced temperature in tissues outside the bronchial wall. The liquid flowing within the balloon 12 may include a radiographic contrast agent to aid in visualization of the balloon and verification of proper placement.

The ultrasound control or energization system 100 uses transducer 11 to measure the size of the bronchus. The control unit 104 and ultrasonic signal generator 106 actuate the transducer 11 to “ping” the bronchus with a low power ultrasound pulse. The ultrasonic waves in this pulse are reflected by the bronchial wall onto transducer 11 as echoes. Transducer 11 converts the circumferentially accumulated (volumetrically integrated) acoustic (ultra-acoustic) echoes to electrical echo signals. The ultrasound system particularly including control unit 104 then determines the size of the bronchus by analyzing the echo signals in a time and amplitude domain, as shown in FIG. 4.

For example, the ultrasound system may measure a time delay between actuation of the transducer 11 to produce the “ping” and the return of echo signals. The width of the return signal represents the difference between a maximum diameter of the reflecting structure, d max, and a minimum diameter d min in case the bronchial section is not perfectly circular but oval shaped. If pump 216 is a pulsating pump, the echo signal is modulated by the pump pulsation and can therewith be differentiated from other stable amplitude/time/phase echoes by its temporal signature. The actuator or control unit can be configured, either with programming or solid state circuits, to process the ultrasound echoes to differentiate bronchial wall echoes from irrelevant returning waveforms.

The ultrasound system uses the measured bronchus size to set the acoustic power to be delivered by transducer 11 during application of therapeutic ultrasonic energy in later steps. For example, the control unit may use a lookup table correlating a particular echo delay (and thus bronchial diameter) with a particular power level as shown in FIG. 7. Generally, the larger the diameter, the more energy is required. Moreover, a pulsating balloon pressure enables one to monitor the effectiveness of smooth muscle ablation. By monitoring the cooling fluid pressure, and in case of a peristaltic pump the pressure and balloon pulsation, the compliance and therewith smooth muscle ablation of the surrounding bronchus can be characterized. If smooth muscle is ablated, the vessel wall tends to be very compliant and follows the pulsation. With intact smooth muscle, the pulsation is damped. Thus, control unit 104 is configured, whether by use of programming in the case of a microprocessor or by virtue of circuit configuration in the case of a specially configured solid state circuit or the use of AI, to detect relaxation of smooth muscle as a result of ultrasound treatment as described herein.

The physician initiates the treatment through a user interface (not shown). In the treatment, the ultrasonic system, and particularly the actuator or control unit 104 and the ultrasonic signal generator 106 energize transducer 11 to deliver therapeutically effective ultrasonic waves to an impact volume 13 (FIG. 2 and FIG. 7). The ultrasound energy emitted by the transducer 11 propagates generally radially outwardly and away from the transducer 11 encompassing a full circle, or 360° of arc about the proximal-to-distal axis of the transducer 11 and the axis of the bronchial section treated. The selected operating frequency, unfocused characteristic, placement, size, and the shape of the ultrasound transducer 11 allow the entire bronchial section and bronchial nerves to lie within the “near field” region of the transducer 11. As shown in FIG. 2, within this region an outwardly spreading, unfocused omni directional (360°) cylindrical beam of ultrasound waves generated by the transducer 11 tends to remain collimated and has an axial length approximately equal to the axial length of the transducer 11. For a cylindrical transducer, the radial extent of the near field region is defined by the expression L2/λ, where L is the axial length of the transducer 11 and λ is the wavelength of the ultrasound waves. At distances from the transducer 11 surface greater than L2/λ, the beam begins to spread axially to a substantial extent. However, for distances less than L2/λ, the beam does not spread axially to any substantial extent (FIG. 2). Therefore, within the near field region, at distances less than L2/λ, the intensity of the ultrasound energy decreases according 1/r as the unfocused beam spreads radially. As used in this disclosure, the term “unfocused” refers to a beam, which does not increase in intensity in the direction of propagation of the beam away from the transducer 11. The impact volume 13 is generally cylindrical and coaxial with the bronchial section treated (FIG. 2). It extends from the transducer outer surface to an impact radius, outside of which the intensity of the ultrasonic energy is too small to heat tissue to a temperature that will cause inactivation of nerves and smooth muscle (see FIG. 7).

As discussed above, the length of the transducer 11 may vary between about 2 mm and about 10 mm but is preferably about 6 mm to provide a wide inactivation zone of the bronchial nerves and smooth muscle. The diameter of the transducer 11 may vary between about 1.5 mm to about 3.0 mm and is preferably about 2.0 mm. Other lengths and diameters are also contemplated. The dosage is selected not only for its therapeutic effect, but also to allow the radius of the impact volume 13 to be between preferably 1 mm and up to a few millimeters depending on bronchial diameter measured from the outer surface of the balloon 12 in order to encompass both the smooth muscle in the bronchial section treated and adjacent bronchial nerves, without transmitting damaging ultrasound energy to collateral structures such as esophagus 3 and peri-esophageal nerves in FIG. 1.

The power level desirably is selected so that throughout the impact volume, solid tissues are heated to about 60° C. or more for several seconds or more, but desirably the wall of the bronchus remains well below 45° C. and preferably below 40° C., as shown in FIG. 7. Thus, throughout the impact region 13, the solid tissues (including all of the bronchial nerves and smooth muscle) are brought to a temperature sufficient to inactivate nerve conduction and damage smooth muscle but below that which causes rapid necrosis of the surrounding tissues.

Research shows that nerve damage occurs at much lower temperatures and much faster than tissue necrosis. See Bunch, Jared. T. et al. “Mechanisms of Phrenic Nerve Injury During Radiofrequency Ablation at the Pulmonary Vein Orifice, Journal of Cardiovascular Electrophysiology, Volume 16, Issue 12, pg. 1318-1325 (Dec. 8, 2005), incorporated by reference herein. Since, necrosis of tissue typically occurs at temperatures of 65° C. or higher for approximately 10 sec or longer while inactivation of nerves typically occurs when the nerves are at temperatures of 42° C. or higher for several seconds or longer, the dosage of the ultrasound energy is chosen to keep the temperature in the impact volume 13 between those temperatures for several seconds or longer as shown in FIG. 7. Operation of the transducer thus provides a therapeutic dosage, which inactivates nerves and ablates smooth muscle without causing further damage to the bronchus and particularly the mucosa at the treatment site. In addition, the circulation of cooled liquid through the balloon 12 containing the transducer 11 may also help reduce the heat being transferred from the transducer 11 to the inner layer of the bronchus. Hence, the transmitted therapeutic unfocused ultrasound energy does not damage the inner layer of the bronchus, providing a safer treatment.

The diagnostic mode can also in some embodiments be utilized to detect water heating or the efficacy of the cooling. Since speed in hot water is faster, the analysis of electrical echo signals in the time domain can assess temperature parameters or ranges. For example, measurement of a time delay between emitting signals and the return of echo signals can be used for temperature assessment or in certain instances temperature measurement if various temperatures can be pre-associated with time or temperature changes if temperature shifts can be pre-associated with shifts in time.

In order to generate the therapeutic dosage of ultrasound energy, the acoustic power output of the transducer 11 typically is approximately 10 watts to approximately 100 watts, more typically approximately 20 to approximately 30 watts. The duration of power application typically is approximately 2 seconds to approximately a minute or more, more typically approximately 10 seconds to approximately 20 seconds (see FIG. 7). The optimum dosage used with a particular system to achieve the desired temperature levels may be determined by mathematical modeling and confirmed by animal testing.

The impact volume 13 of the unfocused ultrasound energy encompasses the entire bronchial section treated and closely surrounding tissues, and hence encompasses all of the smooth muscle and bronchial nerves surrounding the bronchus. Therefore, the placement in the bronchus of the transducer 11 may be indiscriminate in order to inactivate conduction of all the surrounding bronchial nerves 6 surrounding the bronchi in the subject. As used in this disclosure “indiscriminate” and “indiscriminately” mean without targeting, locating, or focusing on any specific bronchial nerves or smooth muscle.

Optionally, the physician may then reposition the catheter 10 and transducer 11 along the bronchus and reinitiate the treatment to retransmit therapeutically effective unfocused ultrasound energy. This inactivates the bronchial nerves and smooth muscle at an additional location along the length of the bronchial tree, and thus provides a more reliable treatment. The repositioning and retransmission steps optionally can be performed multiple times. Next the physician moves the catheter 10 with the transducer 11 to the other lung half (le/ri) and performs the entire treatment again for that bronchial side (see FIG. 6). After completion of the treatment, the catheter 10 is withdrawn from the subject's body.

In some embodiments, ultrasonic energy emitted at a sub-therapeutic level, e.g., a level used for the diagnostic level or another level below the therapeutic level, so further ablation does not occur, can be utilized to detect/assess the result of the tissue (smooth muscle) ablation. Since ablated tissue is more echo reflective as compared to non-ablated tissue, the signals can be processed and analyzed and thus determine the status of ablation, e.g., whether it is complete, the effectiveness, in the target volume. That is, the difference in tissue absorption, which affects the reflected signal, can be detected to determine the ablated state of tissue. Thus, in some embodiments, the system can be utilized to further transmit ultrasound energy at the sub-therapeutic level after transmitting ultrasound energy at the therapeutic level has ablated peri bronchial tissues for procedure assessment.

In some embodiments, the quasi-simultaneous (pulsed therapeutic/diagnostic interleaved) mode discussed herein can be used with smooth muscle ablation detection/assessment so the clinician can assess ablation progress in real time.

Numerous variations and combinations of the features discussed above can be utilized. For example, the ultrasound system may control the transducer 11 to transmit ultrasound energy in a pulsed function instead of a continuous function during application of therapeutic ultrasonic energy. The pulsed function causes the ultrasound transducer 11 to emit the ultrasound energy at a duty cycle of, for example, 50%. Pulse modulation of the ultrasound energy is helpful in limiting the tissue temperature while increasing treatment times. The pulsed therapeutic function can also be interleaved with diagnostic volumetric A mode acquisitions. This way diagnostic ultrasound information can be obtained (quasi)simultaneously to the therapeutic treatment.

In a further variant, the bronchial diameters can be measured by techniques other than actuation of transducer 11 as, for example, by radiographic imaging or magnetic resonance imaging or use of a separate ultrasonic measuring catheter. In this instance, the data from the separate measurement can be used to set the dose. The actuator or control unit can select the power level and duration upon manual input of diametric data.

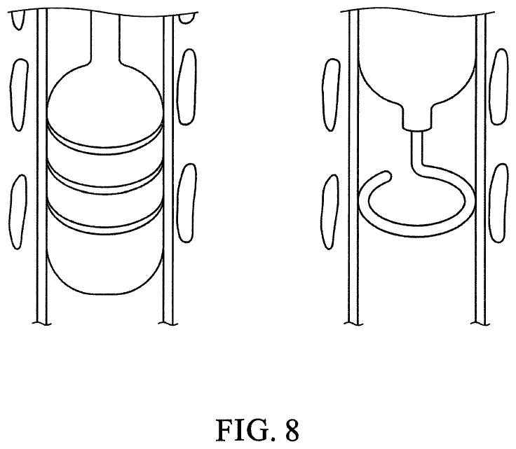

Another variant allows cartilage detection through electrical impedance measurements as shown in FIG. 8A. Cartilage sections have a greater impedance than soft tissues. The outer surface of a balloon 302 is provided with an axially fixed electrode 306 (fixed relative to the balloon). Another electrode 304 is axially movable. The electrodes 304 and 306 preferably take the form of circular bands or rings. FIG. 8A shows electrode 304 as a circularly curled terminal end portion of an electrode member 308. By moving the circular electrode 304 (FIG. 8A) along a bronchus B, cartilage covered sections CS can be identified. In another embodiment, several axially spaced apart circular electrodes 310 are arranged along a balloon 312 (FIG. 8B) and are activated individually by control unit 104 to detect the impedance maximum and therewith cartilage locations. Thus, the actuator or control unit can analyze the impedance measurements to determine locations of bronchial cartilage rings and the actuator or control unit can activate the ultrasound transducer to transmit ultrasound therapeutic waveform energy between adjacent cartilaginous bronchial tissue.

It is to be understood that the cartilage detection system of FIG. 8A or 8B may be used with other forms of treatment energy, for instance, RF. Thus, the system includes an ultrasound transducer 11 (see, e.g., FIG. 1) or an RF energy transducer adapted for insertion into a bronchial branch of a bronchial tree of the mammalian subject and for transmitting energy into surrounding tissue. Actuator or control unit 104 is electrically connected to the transducer 11 (whether ultrasound or other energy) and adapted to control the transducer to emit energy between cartilage locations into an impact volume encompassing the bronchial branch so that the energy is applied at a therapeutic level sufficient to inactivate conduction of bronchial nerves throughout the impact volume. In other words, the use of the electrodes for cartilage detection can be used with the ultrasound diagnostic and treatment systems and methods disclosed herein. The balloon mounted electrodes can be on a separate catheter or part of the same catheter containing the balloon and transducer. If part of the same catheter, it can be mounted on the fluid filled balloon having the transducer inside or can be supported by another balloon distal or proximal of the transducer-containing balloon.

In a further variant, the balloon 12 may be formed from a porous membrane or include holes, such that cooled liquid being circulated within the balloon 24 may escape or be ejected from the balloon 12 against the bronchial walls to improve acoustic contact and mobility. A reduction of friction between the balloon and the bronchial wall is particularly beneficial in axially adjusting the location of the transducer-and-balloon assembly to facilitate ultrasound application between adjacent cartilaginous tissue. This functionality is less important in tertiary and fourth generation bronchi as the cartilage density decreases the further distal in the bronchial tree.

Typically, catheter 10 is a disposable, single-use device. The catheter 10 or ultrasonic system may contain a safety device that inhibits the reuse of the catheter 10 after a single use. Such safety devices per se are known in the art.

In yet another variant, the catheter 10 itself may include a steering mechanism which allows the physician to directly steer the distal end of the catheter. In this case a bronchoscope or sheath may be omitted.

Although the systems, apparatus and methods of the subject invention have been described with reference to particular embodiments, it is to be understood that these embodiments are merely illustrative of the principles and applications of the present invention. It is therefore to be understood that changes and modifications may be made to the illustrative embodiments and that other arrangements may be devised without departing from the spirit and scope of the present invention as defined by the appended claims.

Additionally, persons skilled in the art will understand that the elements and features shown or described in connection with one embodiment may be combined with those of another embodiment without departing from the scope of the present invention and will appreciate further features and advantages of the presently disclosed subject matter based on the description provided.

Throughout the present invention, terms such as “approximately,” “generally,” “substantially,” and the like should be understood to allow for variations in any numerical range or concept with which they are associated. For example, it is intended that the use of terms such as “approximately” and “generally” and “substantially” should be understood to encompass variations on the order of 25%, or to allow for manufacturing tolerances and/or deviations in design.

Although terms such as “first,” “second,” “third,” etc., may be used herein to describe various operations, elements, components, regions, and/or sections, these operations, elements, components, regions, and/or sections should not be limited by the use of these terms in that these terms are used to distinguish one operation, element, component, region, or section from another. Thus, unless expressly stated otherwise, a first operation, element, component, region, or section could be termed a second operation, element, component, region, or section without departing from the scope of the present disclosure.

Each and every claim is incorporated as further disclosure into the specification and represents embodiments of the present disclosure. Also, the phrases “at least one of A, B, and C” and “A and/or B and/or C” should each be interpreted to include only A, only B, only C, or any combination of A, B, and C.

1. System for simultaneously inactivating bronchial nerve conduction and ablating smooth muscle in a mammalian subject, comprising:
an elongated member supporting an ultrasound transducer adapted for insertion into a bronchial branch of a bronchial tree of the mammalian subject and for transmitting ultrasound energy; and
an actuator or control unit electrically connected to the transducer, the actuator or control unit adapted to control the ultrasound transducer to i) transmit ultrasound energy at a sub-therapeutic level in a diagnostic mode; and ii) transmit ultrasound energy in an impact volume encompassing the bronchial branch in a therapeutic mode so that the ultrasound energy is applied at a therapeutic level sufficient to inactivate conduction of bronchial nerves and ablate smooth muscle throughout the impact volume in a therapeutic mode.

2. The system of claim 1, wherein the actuator or control unit transmits ultrasound energy in the diagnostic mode to generate a signal and integrate ultrasound echoes to measure bronchial diameter.

3. The system of claim 1, wherein the actuator or control unit is configured to analyze the impedance measurements to determine locations of bronchial cartilage rings and the actuator or control unit is additionally configured to activate the ultrasound transducer to transmit ultrasound therapeutic waveform energy between adjacent cartilaginous bronchial tissue.

4. The system of claim 1, wherein the actuator or control unit is configured to transmit ultrasound energy in the diagnostic mode to generate a signal and analyze ultrasound echoes to ensure circumferential coupling.

5. The system of claim 1, wherein the actuator or control unit is configured to transmit ultrasound energy in the diagnostic mode to generate a signal and analyze ultrasound echoes to ensure inter-cartilage positioning.

6. The system of claim 1, wherein analysis of the return signal of the transmitted pulse in the diagnostic mode determines one or both of a type or state of tissue.

7. The system of claim 1, wherein analysis of the return signal of the transmitted pulse in the diagnostic mode provides centering of the transducer in the bronchial branch.

8. The system of claim 1, wherein the actuator or control unit is configured to transmit ultrasound energy at the therapeutic level interleaved with transmitting in the diagnostic mode.

9. The system of claim 1, wherein the actuator or control unit is configured to transmit pulsed signals at the therapeutic level.

10. The system of claim 1, wherein the actuator or control unit further transmits ultrasound energy at the sub-therapeutic level after transmitting ultrasound energy at the therapeutic level has ablated peri bronchial tissues.

11. The system of claim 1, further comprising a balloon, the balloon containing cooling fluid.

12. The system of claim 11, wherein pulsating fluid within the balloon enables volumetric A mode diameter measurements by analyzing amplitude and signal width fluctuations.

13. The system of claim 11, wherein the cooling fluid is pulsated to provide pulsating flow to measure bronchial compliance and degree of smooth muscle ablation.

14. The system of claim 11, wherein the transducer emits ultrasonic energy at a sub-therapeutic level to detect heating of the fluid and/or efficacy of cooling.

15. The system of claim 1, further comprising at least one electrode for making impedance measurements along a wall of the bronchial branch to locate cartilaginous tissue and spaces or gaps between cartilaginous tissue.

16. The system of claim 1, wherein the transducer transmits ultrasound energy in the therapeutic mode to direct the ultrasound energy between cartilage rings.

17. System for conducting pulmonary treatment in a mammalian subject, comprising:
an elongated member having a balloon and an energy transducer within the balloon, the transducer adapted for insertion into a bronchial branch of a bronchial tree of the mammalian subject and for transmitting energy into surrounding tissue, and
an actuator or control unit electrically connected to the transducer, the actuator or control unit being adapted to control the transducer to emit ultrasonic energy into an impact volume encompassing the bronchial branch so that the energy is applied at a therapeutic level sufficient to inactivate conduction of bronchial nerves throughout the impact volume;
wherein the balloon contains cooling fluid and the cooling fluid is pulsated to measure bronchial compliance.

18. The system of claim 17, wherein the transducer emits ultrasound energy at a sub-therapeutic level in a diagnostic mode to measure bronchial diameter to optimize dosing.

19. The system of claim 17, wherein the cooling fluid is pulsated to generate an A-mode amplitude modulation to enable echo identification for bronchial diameter measurements.

20. The system of claim 19, wherein the pulsating fluid within the balloon enables volumetric A mode diameter measurements by analyzing amplitude and signal width fluctuations.

21-56. (canceled)
